SEQUENCE LISTING (9 sequences)

SEQ ID NO: 1 - protein, 167 aa, Homo sapiens
MVSNFFHVIQVFEKSATLISKTEHIGFVIYSWRKSTTHLGSRRKFAISIYLSEVSLQKYDCPFSGTSFVVFSLFLICAMAGDVVYADIKTVRTSPLELAFPLQRSVSFNFSTVHKSCPAKDWKVHKGKCYWIAETKKSWNKSQNDCAINNSYLMVIQDITAMVRFNI

SEQ ID NO: 2 - protein, 47 aa, Homo sapiens
DWKVHKGKCYWIAETKKSWNKSQNDCAINNSYLMVIQDITAMVRFNI

SEQ ID NO: 3 - protein, 12 aa, Homo sapiens
IQDITAMVRFNI

SEQ ID NO: 4 - protein, 113 aa, Mus musculus
EVQLQQSGAELVRSGASVKLSCTTSGFNIKDFYIHWVRQRPEQGLEWIGWIDPKNDDTEYAPRFQGKATVTADTSSNTAYLHLSGLTSEDTAVYYCNGRYPTMDFWGQGTSVT

SEQ ID NO: 5 - protein, 108 aa, Mus musculus
EIVLTQSPITMTTSPGEKITITCSASSSISSHYLHWYQQKPGFSPKLLIYRASNLASGVPTRFSGSGSGTSYSLTIDTMEAEDVATYFCQQGSSLPLTFGAGTKLELK

SEQ ID NO: 6 - protein, 122 aa, Mus musculus
EVQLQQSGTVLVRSGASVKLSCTASGFNIKEYYIHWVKQRPEQGLEWIAWIDLENGESEYAPKFQGKATLTADTSSNTAYLQLSGLTSEDTAVYYCNAFHGNYAHNYAIDYWGQGTSVTVSQ

SEQ ID NO: 7 - protein, 112 aa, Mus musculus
DIVMSQSPSSLAVSAGEKVTMTCKSSQSLLNSRTRKNYLAWYQQKPGQSPKLLIYWASTRKSGVPDRFTGSGSGTDFTLTISSVQAEDLAVYYCKQSYNLRTFGGGTKLEIK

SEQ ID NO: 8 - protein, 116 aa, Mus musculus
EVKLLESGGGLVQPGGSLKLSCAASGFDFSRYWMSWVRQAPGKGLEWIGEINPDSSTINYTPSLKDKFIISRDNAKNTLYLQMSKVRSEDTALYYCARRGGYFAYWGQGTLVTVSA

SEQ ID NO: 9 - protein, 108 aa, Mus musculus
DIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWK

(19) United States
(12) Patent Application Publication
(10) Pub. No.: US 2025/0154263 A1
(43) Pub. Date: May 15, 2025

(54) ANTI-CLECL1 ANTIBODIES FOR TREATMENT AND PROGNOSIS OF CHRONIC LYMPHOCYTIC LEUKEMIA AND OTHER DISEASES

(71) Applicant: The Feinstein Institutes for Medical Research, Manhasset, NY (US)

(72) Inventors: Nicholas Chiorazzi, Manhasset, NY (US); Shih-Shih Chen, Manhasset, NY (US); Xiao Jie Yan, Manhasset, NY (US)

(73) Assignee: The Feinstein Institutes for Medical Research, Manhasset, NY (US)

(21) Appl. No.: 18/947,206

(22) Filed: Nov. 14, 2024

Related U.S. Application Data
(60) Provisional application No. 63/548,420, filed on Nov. 14, 2023.

(51) Int. Cl. C07K 16/28; A61P 35/02; G01N 33/68

(52) U.S. Cl. CPC C07K 16/2851; A61P 35/02; G01N 33/6854; C07K 2317/24; C07K 2317/34; C07K 2317/565; G01N 2800/54

(57) ABSTRACT

Methods are disclosed for treating chronic lymphocytic leukemia (CLL), and other diseases, comprising administrating to a patient an anti-CLECL1 antibody or antigen-binding fragment thereof, as soluble molecules or as components of CAR-T cells or as bi- or tetra-specific antibodies that co-ligate T cells or NK cells and abnormal cells in CLL or other diseases or as bi- or tetra-specific antibodies that bind only the B cells in CLL or only abnormal cells in other diseases. Methods are disclosed for determining residual disease after therapy or predicting disease relapse in a patient comprising analyzing a blood or bone sample from a patient treated for the disease for the presence of CLECL1⁺ PF cells using a monoclonal antibody to CLECL1 or antigen-binding fragment thereof, wherein reappearance of CLECL1⁺ PF cells in the blood of the patient is indicative of development of resistance to therapy. Monoclonal antibodies to CLECL1 are disclosed.

### CROSS-REFERENCE TO RELATED APPLICATIONS

This application claims the benefit of U.S. Provisional Patent Application No. 63/548,420, filed on Nov. 14, 2023, the contents of which are herein incorporated by reference in their entirety into the present application.

### INCORPORATION BY REFERENCE OF SEQUENCE LISTING

The Sequence Listing entitled “50425-827.xml”, 9 KB, created on Nov. 12, 2024 and submitted herewith electronically using the U.S. Patent Center is incorporated by reference as the Sequence Listing XML for the subject application.

### BACKGROUND

Throughout this application various publications are referred to in parentheses. Full citations for these may be found at the end of the specification immediately preceding the claims. The disclosures of these publications are hereby incorporated by reference in their entireties into the subject application to more fully describe the art to which the subject application pertains. The discussion of these publications herein is intended merely to summarize the assertions made by Applicant and no admission is made that any publication constitutes prior art.

Chronic lymphocytic leukemia (CLL) is the most common leukemia in western countries, and, despite recent therapeutic advances, remains incurable short of high mortality approaches such as hematopoietic stem cell transplantation or chimeric antigen receptor-T (CAR-T) cell therapy. In addition, although treatment with inhibitors of Bruton's tyrosine kinase (BTKi) and with inhibitors of the pro-survival protein BCL-2 (BCL-2i), alone or in combinations of the two or in combinations with other therapeutic moieties (e.g., monoclonal antibodies, mAbs) improve the quality of life and can abate disease progression, resistance to these therapies develops¹⁻³. Hence, novel effective treatments that do not attack the same pathways are needed.

A major mechanism of action of medications that block the action of Bruton's tyrosine kinase (BTK), e.g., ibrutinib (commercial name, Imbruvica), is the inhibition of CLL cell growth. However, these drugs must be taken continuously at an enormous yearly cost (˜$120,000/year). Thus, the availability of a therapeutic that could be given intermittently would be valuable and probably less costly.

CLL patients experience progression of disease when subclones within the leukemic bulk that carry additional genomic aberrations emerge and expand (“clonal evolution”). Therefore, therapies that abort clonal evolution would prevent or at least delay disease progression in CLL and allied diseases.

Moreover, CLL cells depend on elements in the tissue microenvironment (TME) that produce factors that promote the survival and growth of the leukemia cells. Il-4 is a product of T cells in the TME that can be immunosuppressive, blocking surveillance and elimination of tumor cells, thereby prolonging leukemia cell survival and expansion. So, therapeutic approaches that would prevent the production of IL-4 would be effective in reducing the life span of a leukemic cell as well as improving the ability of a patient's immune system to recognize, respond to and eliminate leukemic cells.

CLECL1 was first described in 2002 as a human “Dendritic Cell (DC) Associated Lectin” (DCAL-1) that was also found on monocytes and some B cells⁴. Although CLECL1 (the current name for DCAL-1) is a C-type lectin-like molecule, it lacks 3 cysteines required to form a standard carbohydrate recognition domain (CRD). Functional analyses indicated that CLECL1 interacts with naïve CD4⁺ T cells, enhances CD3 triggered proliferation, and leads to the development of Th2 cells that make IL-4⁵. The only other study directly addressing CLECL1 as an immunologic agent was carried out by the same team that discovered the molecule; this investigated the role of CLECL1 in DC maturation⁶. So, prior to the studies reported in this application, there were no studies of CLECL1's role in the development and evolution of a B cell lymphoproliferative disorder, including CLL, or of B cells in other diseases in which CLECL1 might be involved.

We have found that CLECL1 is expressed on CLL cells at the time a cell divides and after cell division and are enriched in the portion of the CLL clone that is termed the “proliferative fraction” (PF)⁷. The PF is defined by immunofluorescence using flow cytometry by the presence of the following marker proteins on the leukemia cell surface membrane-CD19⁺CD3⁻ CXCR4^{Dim}CD5^{Bright}. Hence, since the CLECL1⁺ fraction of a CLL cell clone has just divided, it expands tumor burden. Moreover, the DNA mutators, activation-induced cytidine deaminase (AID)⁸ are expressed preferentially in CLECL1⁺ PF cells. While CLECL1⁺ PF cells are a component of the PF, the PF has the highest levels of reactive oxygen species (ROS)⁹ within members of a CLL clone. Also, we have documented that interaction of CLECL1⁺ PF cells with naïve T cells (Tn) leads to Th2 cell development; additionally, interaction of CLECL1 with committed Th2 cells expands these. Both actions result in in the production of IL-4¹⁰.

Thus, eliminating CLECL1⁺ PF cells would remove the fraction of a leukemic clone that recently replicated its DNA to allow cell division. Since DNA replication would have occurred in these cells in the presence of the DNA mutators AID and ROS, these cells could have developed novel genomic mutations that could lead to clonal evolution and ultimately disease progression. Moreover, eliminating CLECL1⁺ PF cells would remove the fraction of a leukemic clone that has the capacity to induce and expand Th2 cells that make IL-4. Since IL-4 has the ability to help CLL cells to survive and grow¹¹ and to prevent the immune system from recognizing and eradicating the leukemic, this would reduce tumor burden, especially those cells that have developed mutations that could allow clonal evolution and promote disease progression.

For clarification, another lectin-like molecule is overexpressed in acute myelogenous leukemia (AML) that was initially referred to as C-type lectin 1. This molecule and CLECL1 have been confused in the past and, on occasion, their biologic properties merged. The molecule that is important in AML is now referred to as CLEC12A.

### SUMMARY OF THE INVENTION

The invention provides a method of treating chronic lymphocytic leukemia (CLL), as well as other diseases, in a patient in need thereof comprising administrating to the patient a therapeutically effective amount of an anti-CLECL1 antibody or antigen-binding fragment thereof that is administered to the patient alone or in combination with a toxin or Chimeric Antigen Receptor (CAR) T cells that bear an anti-CLECL1 mAb or bi- or tetra-specific antibody molecules that co-ligate T cells or NK cells+abnormal cells or that specifically target the abnormal cells by using a lineage-specific reagent plus an anti-CLECL1 mAb.

The present invention provides monoclonal antibody-based therapies to eliminate the fraction of CLL cells that most likely bear new genomic mutations and that capable of leading to a biased Th2-enriched TME by two distinct mechanisms.

The invention also provides a method of measuring the level of residual disease after various therapies, determining if the residual disease is dormant or is growing, and of predicting disease relapse in a patient. The method comprises using one or a series of monoclonal antibodies to CLECL1 or antigen-binding fragment thereof to analyze blood, bone marrow, lymph node, or other tissue samples from a patient who is or has been treated for the disease for the presence of CLECL1⁺ PF cells, wherein reappearance of CLECL1⁺ PF cells in the blood of the patient is indicative of the presence of residual disease, static or increasing, the reemergence of the leukemic clone, and hence the development of resistance to or relapse from therapy. Using anti-CLECL1 mAbs to detect expression of CLECL1 could be done in isolation or in combination with other sets of mAbs or reagents that define the presence of residual disease and/or its outgrowth.

The invention further provides monoclonal antibodies to CLECL1.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1. Only CLECL1⁺ CLL cells induce/expand production of IL-4, and targeting CLECL1 blocks the ability of CLECL1⁺ CLL cells to do so. In vitro culture of T cells alone or T cells plus either CLECL1⁺ or CLECL1⁻ CLL PF cells indicated that CLECL1⁺ PF cells promote/amplify the production of IL-4 by Th2 T cells. All the anti-CLECL1 mAbs decrease the levels of IL-4 produced by T cells activated by CLECL1; mAb 243 and mAb 17 are the most potent in this regard. n=3 patients

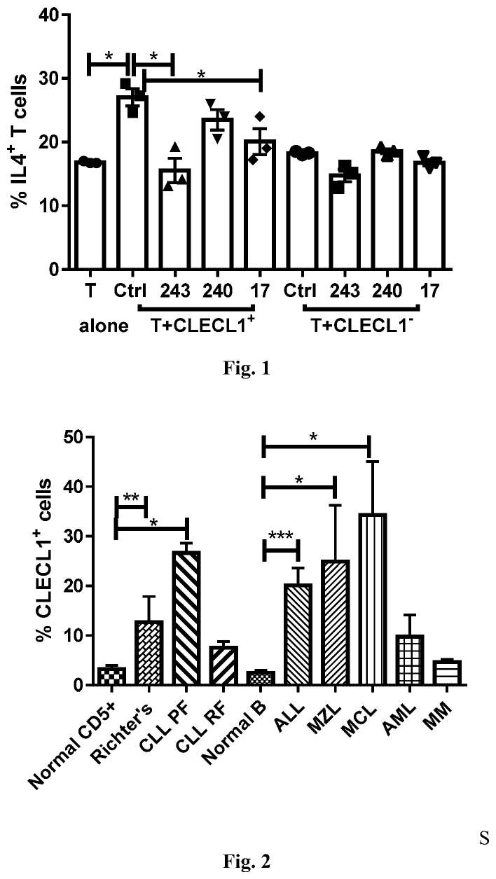

FIG. 2. Significantly higher numbers of CLECL1⁺ PF cells are found in the PF from the blood of patients with CLL than in normal individuals without CLL. This is the case for normal individuals when analyzing unseparated B cells (CD5⁻ and CD5⁺) and purified CD5⁺ B cells. Note that the cells that are resting and that divided previously (“resting fraction”, RF) do not contain significant numbers of CLECL1⁺ PF cells. CLECL1 is also displayed on the surface membranes of several other types of B-cell lymphoproliferative disorders, both acute and chronic, as well as on certain myeloproliferative disorders; this was defined by immunofluorescence using a fluorophore-labeled anti-CLECL1 mAb and measured by flow cytometry. ALL, Acute Lymphocytic Leukemia; MZL, Marginal Zone Lymphoma; MCL, Mantle Cell Lymphoma; AML, Acute Myeloid Leukemia; MM, Multiple Myeloma.

FIG. 3. The numbers of CLECL1⁺ CLL cells is greater in lymph nodes (LNs) than in the peripheral blood (PB). This in consistent with the documentation that more CLL cells divide and grow in LNs than in the bone marrow or the PB¹². Data from paired LN and PB samples from 7 patients with CLL.

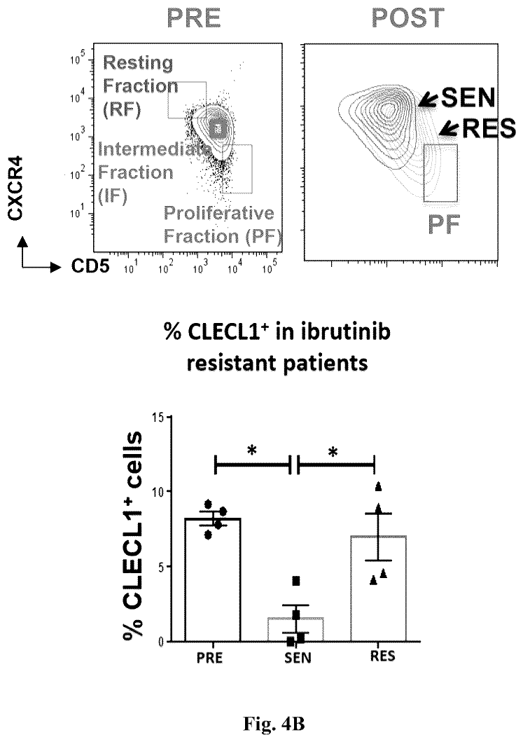

FIG. 4A-4B. The percentages of CLECL1⁺ PF cells associate with time to first treatment (n=133) and with the timing of relapse from ibrutinib/Imbruvica therapy. A. High levels of CLECL1⁺ PF cells in the PF correlate significantly with shorter time to first treatment. Patients have been divided into two categories based on high or low CLECL1⁺ PF cells, using the median. B. BTKi (ibrutinib/Imbruvica in this case) eliminates the PF and the CLECL1⁺ PF cells during the period when the patients were sensitive to the treatment (SEN). However, when the drug is discontinued due to adverse events or the disease becomes resistant to the drug (RES), the CLECL1⁺ PF cells return. N=4 patients treated with ibrutinib/Imbruvica, the first inhibitor of Bruton's tyrosine kinase that was FDA approved.

FIG. 5. Recombinant CLECL1 (rCLECL1) induces naïve T (TN) cells from patients with CLL (n=6) to proliferate and produce IL-4.

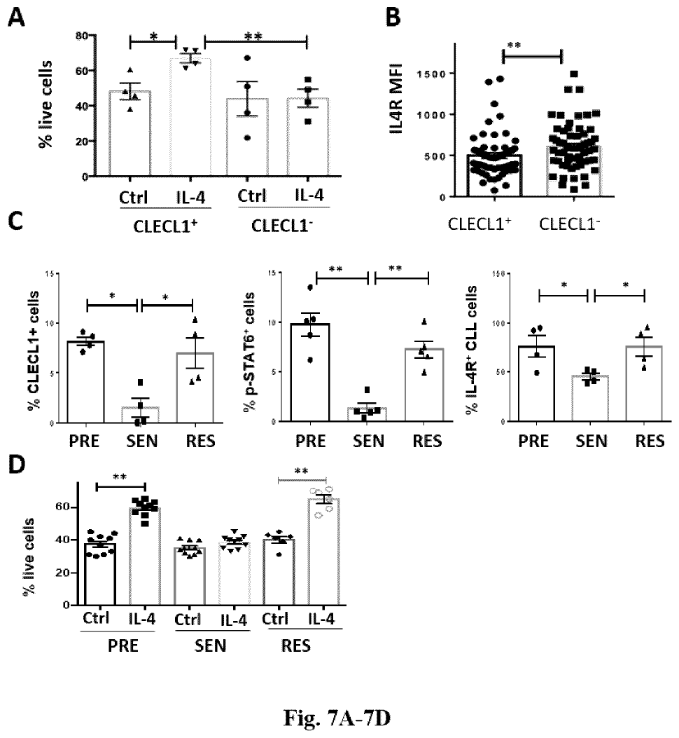

FIG. 6A-6B. A. CLECL1⁺ PF cells induce Th2 T cell expansion in alymphoid NOD.Cg-Prkdc scid Il2rg tm1Wjl/SzJ mice (NSG). B. CLECL1⁺ PF cells stimulate CLL T cells to produce IL-4 in vivo.

FIG. 7A-7D. A. CLECL1⁺, not CLECL1-PF cells respond positively to survival signals delivered by IL-4. B. CLECL1⁺ PF cells express less IL-4R than CLECL1-PF cells. C. Percentage of CLECL1⁺ PF cells, p-STAT6⁺ cells (as an indicator of signaling through the IL-4R), and the numbers of IL-4R+ cells in 4 patients with CLL, collected prior to treatment (PRE) and when the patients' leukemic clones were sensitive (SEN) to and resistant (RES) to ibrutinib/Imbruvica therapy. D. CLL cells collected at the PRE and RES stages, but not the SEN time point, respond positively to IL-4-mediated survival signals in vitro.

FIG. 8A-8F. Generation and validation of murine IgG anti-CLECL1 mAbs. A. Flow cytometry indicates binding of mouse serum taken from a mouse immunized with the 12 C-terminal amino acids of the external domain of CLECL1, linked to KLH, at the time of cell fusion to create hybridomas secreting mAbs reactive with the peptide. B-C. Sorted CLECL1⁺ CLL cells, cultured for 96 hours with autologous CD3⁺ T cells, anti-CD3 mAb, and serum from mouse #4, taken before or after immunization, exhibit significantly reduced (B) CLL cell viability, and (C) IL-4-producing T cells. D-F. Hybridomas #17, 240, and 243 secrete anti-CLECL1 mAbs, documented in (D) ELISA against CLECL1 peptide or rCLECL1 protein and (E) by flow cytometry. Ctrl: PBS only; Positive Ctrl: commercially available agonistic anti-CLECL1 IgM mAb. (F) Anti-CLECL1 mAbs from hybridomas #17, 240, and 243 block the ability of CLECL1⁺ PF cells to induce T cells from a CLL patient to make IL-4-producing Th2 cells.

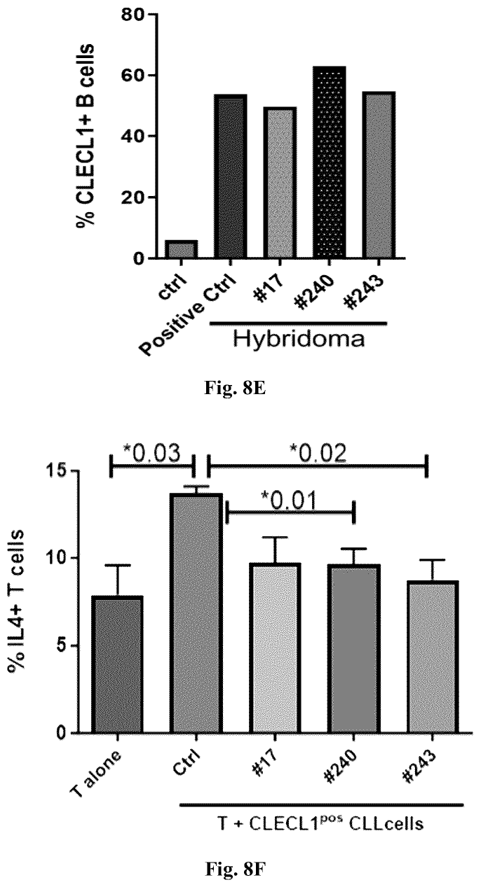

FIG. 9A-9B. CD14⁺ monocytes were cultured alone, in the presence of recombinant CLECL1 (rCLECL1), or with CLECL1⁺ or CLECL1-CLL cells and the numbers and percentages of (A) M2 macrophages, and (B) ratio of M2/M1 macrophages determined.

FIG. 10A-10C. A. Affinity of cold or 37° C. pre-warmed anti-CLECL1 mAbs to the 12-mer peptide determined by ELISA. B. Binding specificities of anti-CLECL1 mAbs to rCLECL1 that was treated (+) or not treated (−) with 2-mercaptoethanol (2-ME) to disrupt intramolecular disulfide bounds and to convert the protein to a primary structure. C. Binding of anti-CLECL1 mAbs to MEC1 cells.

FIG. 11A-11B. A. Abilities of anti-CLECL1 mAbs to carryout complement-dependent cytotoxicity (CDC) and antibody-dependent cellular cytotoxicity (ADCC) on primary patient CLL cells (n=4). Values represent comparisons data of control medium containing guinea pig serum (CDC) or healthy human PBMC (ADCC) with data with each mAb. B. Anti-CLECL1 mAbs elicit antibody-dependent cellular phagocytosis (ADCP) of primary patient CLL cells (n=2) after 24-hour incubation at the ratio of monocyte effector cells to primary CLL target cells indicated.

FIG. 12. NSG mice injected with 10 million MECLI cells were treated with 1 ml of culture supernatants from the hybridomas indicated two times a week for 3 weeks. Treatment of anti-CLELC1 mAbs reduced the absolute number (left) and percentage of Ki-67⁺ (right) MEC1 cells in NSG mouse spleens.

### DETAILED DESCRIPTION OF THE INVENTION

The invention provides methods of treating a disease in a patient in need thereof comprising administrating to the patient a therapeutically effective amount of an anti-CLECL1 antibody or antigen-binding fragment thereof, wherein the disease is one or more of chronic lymphocytic leukemia (CLL), a B-cell lymphoproliferative disorder, follicular lymphoma, Burkitt's lymphoma, mantle cell lymphoma, marginal zone lymphoma, Waldenstrom's macroglobulinemia, diffuse large B-cell lymphoma, multiple myeloma; acute leukemia, acute lymphocytic leukemia, acute myeloid leukemia, acute myelomonocytic leukemia, an autoimmune condition where the production of antibodies by B cells or their derivatives are pathogenic, systemic lupus erythematosus (SLE), rheumatoid arthritis, graft versus host disease (GvHD), multiple sclerosis, a disease where the generation of a Th2 microenvironment is detrimental, atopic dermatitis, asthma, hyper-IgE syndrome, Langerhans cell histiocytosis, a disease where cells of the granulocyte lineage are pathogenic, myelodysplastic syndrome (MDS), and chronic myelogenous leukemia (CML).

In one embodiment, the disease is chronic lymphocytic leukemia (CLL). In one embodiment, the patient is resistant to treatment with an inhibitor of Bruton's tyrosine kinase (BTKi), such as ibrutinib.

In one embodiment, CLECL1 is present on the surface of malignant B cells.

In different embodiments, the antibody or fragment thereof is administered as a component of a CAR-T cell or as a bi- or tetra-specific antibody that co-ligates T cells or NK cells and abnormal cells in the disease or as a bi- or tetra-specific antibody that binds only the B cells in CLL or only abnormal cells in the disease. In different embodiments, the antibody is a component of a CAR-T cell. In different embodiments, the antibody is a component of a bi- or tetra-specific antibody that promotes cytolysis. In different embodiments, the antibody is a component of a a bi- or tetra-specific antibody that enables specific targeting of a cell of a distinct lineage, thereby avoiding off-target toxicity.

In one embodiment, wherein the antibody is a monoclonal antibody. In one embodiment, the antibody or fragment thereof is chimeric or humanized. In one embodiment, the antibody is an IgG monoclonal antibody. In one embodiment, the antibody is an antagonistic monoclonal antibody.

In one embodiment, the antibody is a monoclonal antibody that binds to amino acid sequence DWKVHKGKCY WIAETKKSWN KSQNDCAINN SYLMVIQDIT AMVRENI (SEQ ID NO: 2). In one embodiment, the antibody is a monoclonal antibody that binds to amino acid sequence IQDITAMVRENI (SEQ ID NO:3).

In one embodiment, the antibody is a monoclonal antibody comprising:
a) a heavy chain complementarity determining region (CDR) comprising SEQ ID NO:4,
EVQLQQSGAELVRSGASVKLSCTTSGFNIKDFYIHWVRQRPEQGLEWIGWIDPKN DDTEYAPRFQGKATVTADTSSNTAYLHLSGLTSEDTAVYYCNGRYPTMDFWGQGTSVT, and
a light chain CDR comprising SEQ ID NO:5, EIVLTQSPITMTTSPGEKITITCSASSSISSHYLHWYQQKPGFSPKLLIYRASNLASGVPTRF SGSGSGTSYSLTIDTMEAEDVATYFCQQGSSLPLTFGAGTKLELK; or
b) a heavy chain CDR comprising SEQ ID NO:6, EVQLQQSGTVLVRSGASVKLSCTASGFNIKEYYIHWVKQRPEQGLEWIAWIDLENGESE YAPKFQGKATLTADTSSNTAYLQLSGLTSEDTAVYYCNAFHGNYAHNYAIDYWGQGTS VTVSQ, and a light chain CDR comprising SEQ ID NO:7, DIVMSQSPSSLAVSAGEKVTMTCKSSQSLLNSRTRKNYLAWYQQKPGQSPKLLIYWAST RKSGVPDRFTGSGSGTDFTLTISSVQAEDLA VYYCKQSYNLRTFGGGTKLEIK; or
c) a heavy chain CDR comprising SEQ ID NO:8, EVKLLESGGGLVQPGGSLKLSCAASGFDFSRYWMSWVRQAPGKGLEWIGEINPDSSTIN YTPSLKDKFIISRDNAKNTLYLQMSKVRSEDTALYYCARRGGYFAYWGQGTLVTVSA, and a light chain CDR comprising SEQ ID NO:9, DIVLTQSPASLAVSLGQRATISYRASKSVSTSGYSYMHWNQQKPGQPPRLLIYLVSNLES GVPARFSGSGSGTDFTLNIHPVEEEDAATYYCQHIRELTRSEGGPSWK.

In one embodiment, the antibody or fragment thereof is administered to the patient by intravenous injection.

In one embodiment, the patient is a human or a veterinary animal.

The invention provides methods of treating a disease in a patient in need thereof comprising administrating to the patient a therapeutically effective amount of one or a series of an anti-CLECL1 antibodies or antigen-binding fragment thereof, which are given to a patient alone, or linked to a toxin, or are incorporated into CAR-T cells bearing an anti-CLECL1 mAb or incorporated into bi- or tetra-specific antibody molecules that co-ligate T cells or NK cells+the abnormal cells or a bi-specific antibody that specifically target the abnormal cells by using a lineage-specific reagent+one of the anti-CLECL1 mAbs.

The invention provides methods of treating a disease in a patient in need thereof comprising administrating to the patient a therapeutically effective amount of one or three distinct an anti-CLECL1 antibodies or antigen-binding fragments thereof. The invention provides methods of treating a disease in a patient in need thereof comprising administering to the patient a mAb reactive with T cells or other effector cells and therapeutically effective CAR-T cells comprising one of the three distinct an anti-CLECL1 antibodies or antigen-binding fragments thereof. The invention provides methods of treating a disease in a patient in need thereof comprising administering to the patient a bi- or tetra-specific antibody comprising a mAb reactive with T cells or another effector cell and a mAb that is one of three distinct an anti-CLECL1 antibodies or antigen-binding fragments thereof. The invention provides methods of treating a disease in a patient in need thereof comprising administering to the patient a bi-specific or tetra-specific antibody comprised of one (bispecific) or two (tetra-specific) mAbs reactive with the cell lineage of an abnormal cell and a mAb that is one of three an anti-CLECL1 antibodies or antigen-binding fragments thereof.

The disease can be, for example, one or more of [1] B-cell lymphoproliferative disorder, such as but not limited to CLL, follicular lymphoma, Burkitt's lymphoma, mantle cell lymphoma, marginal zone lymphoma, Waldenstrom's macroglobulinemia, diffuse large B-cell lymphoma, and multiple myeloma; [2] acute leukemia such as but not limited to acute lymphocytic leukemia, acute myeloid leukemia, or acute myelomonocytic leukemia; [3] an autoimmune condition where the production of antibodies by B cells or their derivatives, e.g., plasmablasts and plasma cells, are pathogenic, such as but not limited to systemic lupus erythematosus (SLE), rheumatoid arthritis, graft versus host disease (GvHD) or multiple sclerosis; [4] a disease where the generation of a Th2 microenvironment is detrimental, such as but not limited to atopic dermatitis, asthma, hyper-IgE syndrome, Langerhans cell histiocytosis; and [5] a disease where cells of the granulocyte lineage are pathogenic, such as but not limited to myelodysplastic syndrome (MDS), and chronic myelogenous leukemia (CML).

In one embodiment, the disease is CLL. The patient with CLL may have not been previously treated or might have been treated unsuccessfully or might have relapsed when the leukemia cells become resistant to treatment. Examples of such treatments include but are not limited to inhibitors of Bruton's tyrosine kinase (BTKi), such as the FDA approved ibrutinib (Imbruvica), acalabrutinib (Calquence), zanobrutinib (Zanubrutinib) and pirtobrutinib (Jaypirca); and inhibitors of other signaling pathways such as the anti-apoptotic molecule BCL-2, where upon unresponsiveness or relapse the growth of the abnormal cells returns or becomes accelerated. Other conditions include development of a more virulent disease such as accelerated CLL¹³ or Richter's transformation¹⁴, where the rate of cell proliferation has increased.

The therapeutic approach would be especially valuable when patients discontinue treatment with any of the above treatments because of intolerance of side effects. This occurs in 62.5 per 1000 patient-months for all discontinuers and 32.9 per 1000 patient-months treated with ibrutinib/Imbruvica after ˜2+ years because of intolerable adverse reactions¹⁵. Thus, discontinuance because of lack of efficacy or intolerance of adverse effects creates a large unmet need that is addressed by our approach.

The invention also provides methods for predicting disease relapse in a patient or for measuring residual disease in a patient, the methods comprising analyzing a sample from blood, bone marrow or a lymph node of a patient who is or has been treated for the disease for the presence of CLECL1+ cells, wherein the presence of CLECL1+ cells in the patient's sample is detected using a monoclonal antibody to CLECL1 or antigen-binding fragment thereof, wherein appearance of CLECL1+ cells in the sample is indicative of development of resistance to therapy or presence of residual disease, and wherein the disease is one or more of chronic lymphocytic leukemia (CLL), a B-cell lymphoproliferative disorder, follicular lymphoma, Burkitt's lymphoma, mantle cell lymphoma, marginal zone lymphoma, Waldenstrom's macroglobulinemia, diffuse large B-cell lymphoma, multiple myeloma; acute leukemia, acute lymphocytic leukemia, acute myeloid leukemia, acute myelomonocytic leukemia, an autoimmune condition where the production of antibodies by B cells or their derivatives are pathogenic, systemic lupus erythematosus (SLE), rheumatoid arthritis, graft versus host disease (GvHD), multiple sclerosis, a disease where the generation of a Th2 microenvironment is detrimental, atopic dermatitis, asthma, hyper-IgE syndrome, Langerhans cell histiocytosis, a disease where cells of the granulocyte lineage are pathogenic, myelodysplastic syndrome (MDS), and chronic myelogenous leukemia (CML).

The invention provides methods of determining the presence and level of measurable residual disease during or upon cessation of successful treatment, based on the levels of CLECL1⁺ PF cells in the PF. The invention also provides a method to determine the level of activity of the residual disease, i.e., dormant or growing, based on the levels of CLECL1⁺ PF cells in the PF.

The invention provides methods of predicting relapse based on defining increases in the levels of CLECL1⁺ PF cells in the PF of a CLL patient's leukemic clone over time.

The invention provides methods of predicting disease relapse in a patient, the method comprising analyzing samples from the blood, bone marrow, lymph node, or other anatomic site from a patient who is or has been treated for the disease for the presence of CLECL1+PF cells.

The invention provides methods of determining the presence and level of measurable residual disease during or after cessation of successful treatment. This could involve determining the levels of CLECL1+PF cells in the PF alone (CD19+CD3−CXCR4DimCD5BrightCLECL1+) or in conjunction with established techniques (e.g., CD19, CD20, CD5, CD43, CD79b, CD81, CD22, CD3).

The invention provides methods of determining the level of activity of the disease that is measurable during or after cessation of therapy because of apparent effectiveness, i.e., is the remaining, measurable disease dormant (cells are not dividing) or growing (cells are dividing), based on the levels of CLECL1+PF cells.

The presence of CLECL1+PF cells in the patient's blood, bone marrow or lymph node can be detected using a monoclonal antibody to CLECL1 or antigen-binding fragment thereof. In one embodiment, the reappearance of CLECL1+PF cells in the blood, bone marrow or lymph node of the patient is indicative of development of resistance to therapy. The disease can be one or more of [1] B-cell lymphoproliferative disorder, such as but not limited to CLL, follicular lymphoma, Burkitt's lymphoma, mantle cell lymphoma, marginal zone lymphoma, Waldenstrom's macroglobulinemia, diffuse large B-cell lymphoma, and multiple myeloma; [2] acute leukemia such as but not limited to acute lymphocytic leukemia, acute myeloid leukemia, or acute myelomonocytic leukemia; [3] an autoimmune condition where the production of antibodies by B cells or their derivatives, e.g., plasmablasts and plasma cells, are pathogenic, such as but not limited to systemic lupus erythematosus (SLE), rheumatoid arthritis, graft versus host disease (GvHD) or multiple sclerosis; [4] a disease where the generation of a Th2 microenvironment is detrimental, such as but not limited to atopic dermatitis, asthma, hyper-IgE syndrome, Langerhans cell histiocytosis; and [5] a disease where cells of the granulocyte lineage are pathogenic, such as but not limited to myelodysplastic syndrome (MDS) and chronic myelogenous leukemia (CML).

As used herein, the term “antibody” refers to complete, intact antibodies. Antibody fragments include Fab, Fab′, F(ab′)2, and other fragments thereof which fragments bind the antigen of interest, in this case CLECL1. Complete, intact antibodies include, but are not limited to, monoclonal antibodies such as murine monoclonal antibodies, polyclonal antibodies, chimeric antibodies, human antibodies, and humanized antibodies. These antibodies can be made by cell fusions or recombinant technology directly or after immunizing mice, rats, rabbits, llamas, lampreys, or other species with the entire CLECL1 molecule, solely its internal or external domains or its transmembrane domain, or peptides taken from these regions.

In one embodiment, the antibody is a monoclonal antibody.

In one embodiment, the antibody or fragment thereof is chimeric or humanized.

Various forms of antibodies may be produced using standard recombinant DNA techniques¹⁶. For example, “chimeric” antibodies may be constructed, in which the antigen binding domain from an animal antibody is linked to a human constant domain (an antibody derived initially from a nonhuman mammal in which recombinant DNA technology has been used to replace all or part of the hinge and constant regions of the heavy chain and/or the constant region of the light chain, with corresponding regions from a human immunoglobulin light chain or heavy chain) (see, e.g., Cabilly et al., U.S. Pat. No. 4,816,567, and reference¹⁷. Chimeric antibodies reduce the immunogenic responses elicited by animal antibodies when used in human clinical treatments. In addition, recombinant “humanized” antibodies may be synthesized. Humanized antibodies are antibodies initially derived from a nonhuman mammal in which recombinant DNA technology has been used to substitute some or all of the amino acids not required for antigen binding with amino acids from corresponding regions of a human immunoglobulin light or heavy chain. That is, they are chimeras comprising mostly human immunoglobulin sequences into which the regions responsible for specific antigen-binding have been inserted (see, e.g., PCT patent application WO 94/04679). Animals are immunized with the desired antigen, the corresponding antibodies are isolated and the portion of the variable region sequences responsible for specific antigen binding are removed. The animal-derived antigen binding regions are then cloned into the appropriate position of the human antibody genes in which the antigen binding regions have been deleted. Humanized antibodies minimize the use of heterologous (inter-species) sequences in antibodies for use in human therapies and are less likely to elicit unwanted immune responses. Primatized antibodies can be produced similarly.

Another embodiment of the antibodies employed in the compositions and methods of the invention is a human antibody, which can be produced in nonhuman animals, such as transgenic animals harboring one or more human immunoglobulin transgenes. Such animals may be used as a source for splenocytes for producing hybridomas, as is described in U.S. Pat. No. 5,569,825.

Antibody fragments and univalent antibodies may also be used in the methods and compositions of this invention. Univalent antibodies comprise a heavy chain/light chain dimer bound to the Fc (or stem) region of a second heavy chain. “Fab region” refers to those portions of the chains which are roughly equivalent, or analogous, to the sequences which comprise the Y branch portions of the heavy chain and to the light chain in its entirety, and which collectively (in aggregates) have been shown to exhibit antibody activity. A Fab protein includes aggregates of one heavy and one light chain (commonly known as Fab′), as well as tetramers which correspond to the two branch segments of the antibody Y, (commonly known as F(ab)2), whether any of the above are covalently or non-covalently aggregated, so long as the aggregation is capable of specifically reacting with a particular antigen or antigen family.

In one embodiment, the antibody is an IgG monoclonal antibody.

In one embodiment, the antibody is an antagonistic monoclonal antibody that directly kills or blocks the growth of CLECL1⁺ leukemic cells.

In one embodiment, the antibody inhibits CLECL1⁺ PF cells from inducing naïve T cells to become Th2 cells or to increase the numbers of existing Th2 cells, in each instance preventing the production and limiting the release of the immunosuppressive cytokine IL-4.

In one embodiment, the antibody is a component of a patient's autologous T cells that have been manipulated to express the antibody, usually as a single-chain variable fragment domain (scFv), on the T-cell's surface membrane linked to the linked with gamma or zeta chains of the CD3 molecule^{18, 19}. This provides the T cell with the ability to interact with target cells expressing the antigen of interest and for which the antibody is reactive. Such T cells are referred to as chimeric antigen receptor T (CAR-T) cells. These engineered CAR-T cells kill target cells independent of histocompatibility constraints and the requirements for antibody-dependent and complement-dependent cytotoxicity. The introduction of T cell co-stimulatory domains such as CD28 or 41BB have increased the efficiency and efficacy of function²⁰⁻²². CAR-T therapy has had dramatic results in selected relapsed/refractory CLL patients^{23, 24}, and larger studies indicate beneficial responses in ˜30-50% of patients²⁵⁻²⁷. Currently, there are three CAR-T approaches approved for B-cell disorders:
For multiply (≥2 times) relapsed/refractory CLL: Lisocabtagene Maraleucel (Liso-cel, Breyanzi)
For second line treatment for high-risk Diffuse Large B-cell Lymphoma (DLBCL): Lisocabtagene Maraleucel (Liso-cel, Breyanzi).
For second line treatment for high-risk DLBCL: Axicabtagene ciloleucel (Yescarta).

In one embodiment, the antibody is a component of a bi- or tetra-specific antibody that targets different antigens on cells of different lineages, specifically a signaling molecule on an effector cell and a molecule on the target cell. This promotes the physical interaction of the effector and target cell, the activation of the effector cell, and the elimination of the target cell. As an example of this embodiment but not limited to this example, the variable domains of an antibody reactive with the CD3 molecule, or one its components, on a T cell are recombinantly linked with the variable domains of one of the series of anti-CLECL1 mAbs, leading to an approximation of the two cells, the activation of the T cell, and the elimination of the CLECL1-expressing target cell. Several bi-specific antibodies have been approved by the FDA. These include:
For relapsed/refractory multiple myeloma: Teclistamab, Elranatamab, and Talquetamab
For multiply (≥3 times) relapsed/refractory DLCBL: Glofitamab and Eprcoritamab.

In one embodiment, the antibody is a component of a bi- or tetra-specific antibody that targets two different antigens on the same target cell. This addresses cytolytic activities selectively to the target of interest, thereby avoiding off target unacceptable adverse reactions. As an example of this embodiment but not limited to this example, the variable domains of an antibody reactive with a B lymphocyte or an antibody selectively reactive with the target cell (e.g., anti-idiotypic antibody), are recombinantly linked with the variable domains of one of the series of anti-CLECL1 mAbs, leading to discriminatory binding to the target cell, thereby allowing elimination by apoptosis, complement-dependent cytotoxicity (CDC), antibody-dependent cellular cytotoxicity (ADCC), or antibody-dependent cellular phagocytosis (ADCP). We have shown in a pre-clinical model the efficacy and safety of a bi-specific antibody reactive with the CD20 and CD52 proteins on primary CLL cells²⁸.

Both CAR-T cells and bi-specific antibodies have significant therapeutic efficacy in several types of B-cell lymphoproliferative disorders²⁹.

Human CLECL1 has the following amino acid sequence (SEQ ID NO:1): MVSNFFHVIQ VFEKSATLIS KTEHIGFVIY SWRKSTTHLG SRRKFAISIY LSEVSLQKYD CPFSGTSFVV FSLFLICAMA GDVVYADIKT VRTSPLELAF PLQRSVSFNF STVHKSCPAK DWKVHKGKCY WIAETKKSWN KSQNDCAINN SYLMVIQDIT AMVRENI

The bolded, underlined portion is the external domain (SEQ ID NO:2): DWKVHKGKCY WIAETKKSWN KSONDCAINN SYLMVIQDIT AMVRFNI.

In one embodiment, the antibody is a monoclonal antibody that binds any portion of the external domain of CLECL1.

In another embodiment, the antibody is a monoclonal antibody that reacts with the 12 amino acids at the C′ end of the external domain: IQDITAMVRENI (SEQ ID NO:3).

Anti-CLECL1 antibodies are known. Rabbit anti-human CLECL1 polyclonal antibodies are available from several sources including LSBio (Seattle, WA), Creative Diagnostics (Shirley NY), and Thermo Fisher Scientific. A mouse monoclonal IgG anti-CLECL1 antibody is available from MyBioSource, Inc. (San Diego, CA). Additionally, a mouse monoclonal IgM anti-CLECL1 antibody (UW50) has been reported⁵ and is available from Sigma-Aldrich (St. Louis, MO) and Santa Cruz Biotech (Dallas, TX).

In one embodiment, the antibody or fragment thereof is administered to the patient by intravenous or intramuscular or subcutaneous injection.

The patient can be a human or a veterinary animal.

The invention also provides methods of measuring surface level of CLECL1 on malignant B cells as a prognostic marker in those patients with high percentages of CLECL1⁺ PF cells in the PF.

The invention also provides a method to predict disease relapse in a human or a veterinary animal carrying the disease based on the return of or the increase in the numbers of CLECL1⁺ PF cells on or after treatment. The method comprises analyzing blood, bone marrow, lymph node, and samples from other tissues or anatomic site the numbers of CLECL1⁺ PF cells (CD19⁺CD3⁻ CXCR4^{Dim}CD5^{Bright}CLECL1⁺) at one or at serial time points.

The invention also provides methods to measure the level of residual disease after various therapies and to determine the level of activity of the measured residual disease The method comprises analyzing blood, bone marrow, lymph node, and samples from other tissues or anatomic sites for the numbers of CLECL1⁺ PF cells at one or at serial time points. This would involve the use of flow cytometry or similar technologies to determine the numbers of CD19⁺CD3⁻ CXCR4^{Dim}CD5^{Bright}CLECL1⁺ cells or adding one or a series of anti-CLECL1⁺ mAbs to a standard panel of mAbs (e.g., CD19, CD20, CD5, CD43, CD79b, CD81, CD22, CD3) to define surface membrane analyses to determine the presence of measurable residual disease using flow cytometry^{30, 31}.

The presence of CLECL1⁺ PF cells in, e.g., the patient's blood can be detected using a monoclonal antibody to CLECL1 or antigen-binding fragment thereof,

The disease can be, for example, one or more of [1] B-cell lymphoproliferative disorder, such as but not limited to CLL, follicular lymphoma, Burkitt's lymphoma, mantle cell lymphoma, marginal zone lymphoma, Waldenstrom's macroglobulinemia, diffuse large B-cell lymphoma, and multiple myeloma; [2] acute leukemia such as but not limited to acute lymphocytic leukemia, acute myeloid leukemia, or acute myelomonocytic leukemia; [3] an autoimmune condition where the production of antibodies by B cells or their derivatives, e.g., plasmablasts and plasma cells, are pathogenic, such as but not limited to systemic lupus erythematosus (SLE), rheumatoid arthritis, graft versus host disease (GvHD) or multiple sclerosis; [4] a disease where the generation of a Th2 microenvironment is detrimental, such as but not limited to atopic dermatitis, asthma, hyper-IgE syndrome, Langerhans cell histiocytosis; and [5] a disease where cells of the granulocyte lineage are pathogenic, such as but not limited to myelodysplastic syndrome (MDS), and chronic myelogenous leukemia (CML).

In one embodiment, the disease is chronic lymphocytic leukemia (CLL).

In one embodiment, CLECL1 is present on the surface of malignant B cells.

In one embodiment, the antibody or fragment is chimeric or humanized.

In one embodiment, the antibody is an IgG monoclonal antibody.

In one embodiment, the antibody is a monoclonal antibody that binds to any portion of the transmembrane or external domains of the CLECL1 protein or to the amino acid sequence at the C-terminus of the CLECL1external domain, i.e., IQDITAMVRENI (SEQ ID NO:3) . . . . The antibody can be labeled with a label, such as, for example, a fluorescent, radioactive, or enzyme label. The antibody can also be linked to a cytotoxin.

In one embodiment, the antibody is a monoclonal antibody comprising:
a) a heavy chain complementarity determining region (CDR) comprising SEQ ID NO:4, EVQLQQSGAELVRSGASVKLSCTTSGFNIKDFYIHWVRQRPEQGLEWIGWIDPKNDDTE YAPRFQGKATVTADTSSNTAYLHLSGLTSEDTAVYYCNGRYPTMDFWGQGTSVT, and a light chain CDR comprising SEQ ID NO:5, EIVLTQSPITMTTSPGEKITITCSASSSISSHYLHWYQQKPGFSPKLLIYRASNLASGVPTRF SGSGSGTSYSLTIDTMEAEDVATYFCQQGSSLPLTFGAGTKLELK; or
b) a heavy chain CDR comprising SEQ ID NO:6, EVQLQQSGTVLVRSGASVKLSCTASGFNIKEYYIHWVKQRPEQGLEWIAWIDLENGESE YAPKFQGKATLTADTSSNTAYLQLSGLTSEDTAVYYCNAFHGNYAHNYAIDYWGQGTS VTVSQ, and a light chain CDR comprising SEQ ID NO:7, DIVMSQSPSSLAVSAGEKVTMTCKSSQSLLNSRTRKNYLAWYQQKPGQSPKLLIYWAST RKSGVPDRFTGSGSGTDFTLTISSVQAEDLAVYYCKQSYNLRTFGGGTKLEIK; or
c) a heavy chain CDR comprising SEQ ID NO:8, EVKLLESGGGLVQPGGSLKLSCAASGFDFSRYWMSWVRQAPGKGLEWIGEINPDSSTIN YTPSLKDKFIISRDNAKNTLYLQMSKVRSEDTALYYCARRGGYFAYWGQGTLVTVSA, and a light chain CDR comprising SEQ ID NO:9, QAVVTQESALTTSPGGTVILTCRSSTGAVTTSNYANWVQEKPDHLFTGLIGGTSNRAPG VPVRFSGSLIGDKAALTITGAQTEDDAMYFCALWYSTHYVFGGGTKVTVL

In different embodiments, the monoclonal antibody can be chimeric or humanized or can be a component of a CAR-T cell or a bispecific antibody that co-ligates a T cell or an NK cell with the CLECL1⁺ target cell or a bi- or tetra-specific antibody that is binds to a single cell type or cell linage using a cell type or cell lineage reagent and an anti-CLECL1 antibody.

The invention further provides monoclonal antibodies to CLECL1, where the monoclonal antibodies comprise:
a) a heavy chain complementarity determining region (CDR) comprising SEQ ID NO:4, EVQLQQSGAELVRSGASVKLSCTTSGFNIKDFYIHWVRQRPEQGLEWIGWIDPKNDDTE YAPRFQGKATVTADTSSNTAYLHLSGLTSEDTAVYYCNGRYPTMDFWGQGTSVT, and a light chain CDR comprising SEQ ID NO:5, EIVLTQSPITMTTSPGEKITITCSASSSISSHYLHWYQQKPGFSPKLLIYRASNLASGVPTRF SGSGSGTSYSLTIDTMEAEDVATYFCQQGSSLPLTFGAGTKLELK; or
b) a heavy chain CDR comprising SEQ ID NO:6, EVQLQQSGTVLVRSGASVKLSCTASGFNIKEYYIHWVKQRPEQGLEWIAWIDLENGESE YAPKFQGKATLTADTSSNTAYLQLSGLTSEDTAVYYCNAFHGNYAHNYAIDYWGQGTS VTVSQ, and a light chain CDR comprising SEQ ID NO:7, DIVMSQSPSSLAVSAGEKVTMTCKSSQSLLNSRTRKNYLAWYQQKPGQSPKLLIYWAST RKSGVPDRFTGSGSGTDFTLTISSVQAEDLA VYYCKQSYNLRTFGGGTKLEIK; or
c) a heavy chain CDR comprising SEQ ID NO:8, EVKLLESGGGLVQPGGSLKLSCAASGFDFSRYWMSWVRQAPGKGLEWIGEINPDSSTIN YTPSLKDKFIISRDNAKNTLYLQMSKVRSEDTALYYCARRGGYFAYWGQGTLVTVSA, and a light chain CDR comprising SEQ ID NO:9, QAVVTQESALTTSPGGTVILTCRSSTGAVTTSNYANWVQEKPDHLFTGLIGGTSNRAPG VPVRFSGSLIGDKAALTITGAQTEDDAMYFCALWYSTHYVFGGGTKVTVL.

In different embodiments, the monoclonal antibody can be chimeric or humanized or can be a component of a CAR-T cell or a bi- or tetra-specific antibody that ligates a T cell or another effector cells with the CLECL1⁺ target cell or a bi- or tetra-specific antibody that binds to a single cell type or cell linage using a cell type or cell lineage specific reagent and an anti-CLECL1 antibody, thereby avoiding off-target toxicity.

The monoclonal antibody can be labeled with a fluorescent, radioactive, or enzyme label or linked to a cytotoxin.

### EXPERIMENTAL DETAILS

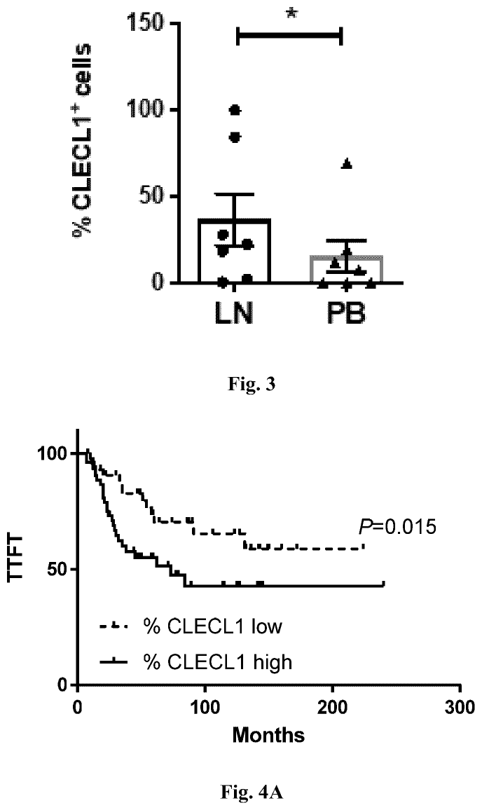

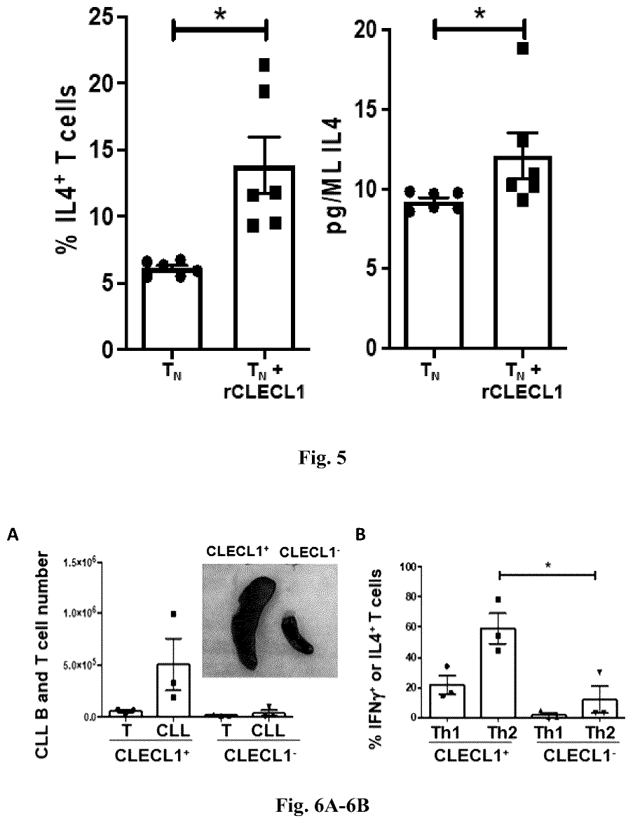

Flow cytometry analyses of CD19⁺CD5⁺ cells from 7 normal people and 77 patients with CLL revealed significantly higher percentages of CLECL1⁺ PF cells in CLL (CLL vs Normal CD19⁺CD5⁺=26.5% vs 3.2%, P=0.002) (FIG. 2). This suggests that there are more B lymphocytes which have recently divided, and hence bear CLECL1, generate DNA mutators AID and ROS in patients with CLL and other related diseases than in normal healthy people. CLECL1 was found most in the fraction of leukemic clones enriched in recently divided cells (“proliferative fraction”, PF; CXCR4^{Dim}CD5^{Bright}) and least in the resting fraction (RF; CXCR4^{Bright} CD5^{Dim}) (PF vs RF=26.7% vs 7.5%, P<0.001). Importantly, high percentages of CLECL1 in the PF correlated with shorter time to first treatment (P=0.01) (FIG. 4A). Notably, effective treatment with ibrutinib significantly reduced the numbers of circulating CLECL1⁺ PF cells (pre-vs post-treatment: 9.1% vs 1.8%, P=0.01), and these levels increased to the pre-treatment levels when patients developed ibrutinib/Imbruvica resistance (sensitive vs resistance=1.8% vs 7.5%, P<0.05).

Flow cytometry analyses also indicated that, compared to CLECL1-CLL cells, CLECL1⁺ PF cells expressed higher levels of other cell interaction molecules such as CD40 and CD80, as well as activation markers, CD23, CD27, CD69, CD71, and CD72. Consistent with this, CLECL1⁺ PF cells were mainly in the G2/M phase of the cell cycle and expressed AID¹⁰. In contrast, CLECL1⁻ PF cells were AID⁻ and chiefly in G1/S phases or undergoing apoptosis¹⁰. Single cell RNAseq analyses of CLL PF versus RF cells corroborated that the expression of CLECL1 is [1] restricted to the PF, [2] associated with genes expressed in G2/M, and [3] correlated with IL-4-related gene expression (not shown). Together, these data indicate that CLECL1⁺ CLL cells are activated B cells generated from CLECL1⁻ cells. Indeed, stimulation of CLECL1⁻ cells anti-IgM+IL-4 in vitro converted CLECL1⁻ to CLECL1⁺ PF cells.

In patient lymph nodes, CLECL1⁺ PF cells expressed Ki67, indicating that the cells are in the active stages of the cell cycle and are dividing or have just divided and co-localized with T cells¹⁰. Therefore, we checked if CLECL1⁺ and CLECL1⁻ cells could interact with autologous T cells and affect T cell growth and cytokine production. Co-culture of unseparated T cells with CLECL1⁺, but not CLECL1⁻ cells, significantly enhanced T cell proliferation upon co-stimulation through T-cell receptors (CLECL1⁺ vs CLECL1⁻: 12.9% vs 2.8%, P=0.02) and induced increased production of IL-4 (CLECL1⁺ vs CLECL1⁻: 15% vs 7%, P<0.05). Published results suggest that recombinant CLECL1 (rCLECL1) only binds to healthy Tn⁵. However, we have found that rCLECL1 directly binds to both Tn and T memory (Tmem) cells from patients with CLL¹⁰. Moreover, when Tn and Tmem cells were incubated with rCLECL1, both T cell subsets responded with enhanced proliferation and IL-4 production¹⁰.

Subsequently, the above in vitro findings were recapitulated in vivo by transferring primary CLL B and T cells into NOD.Cg-Prkdc scid Il2rg tm1Wjl/SzJ (NSG) mice, who are immune deficient because of the lack of T, B, and NK cells. Injection of such alymphoid mice with CLL-derived CD3⁺ T cells plus autologous CLECL1⁺ or CLECL1⁻ PF cells led to significantly higher numbers of CLL B and T cells in the spleens of recipient mice (CLECL1⁺ vs CLECL1⁻ for B cells: 508,800 vs 37,000; P=0.03; for T cells: 273,700 vs 47,000; P=0.03). Additionally, Th2 cells outgrew Th1 T cells (CLECL1⁺ vs CLECL1⁻ for Th2 cells: 60.1% vs 12%; P<0.05; for Th1 cells: 20.1% vs 1.8%; N. S). The same results were seen when NSG mice were injected with CLECL1⁺ or CLECL1⁻ CLL PF cells plus autologous Tn or Tmem cells, as only CLECL1⁺ PF cells stimulated Tn or Tmem cells to expand and produce IL-4. In turn, these CLECL1-stimulated T cells facilitated the growth of CLL B cells in recipient spleen and bone marrow¹⁰.

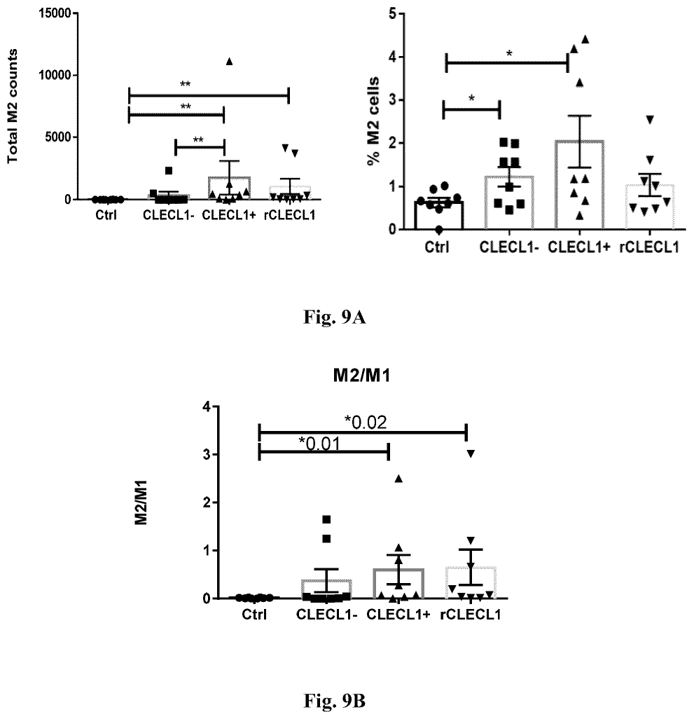

Moreover, the CLECL1⁺ PF as well as recombinant CLECL1 (rCLECL1) protein increased the numbers of M2 macrophages and the ratio of M2 to M1 cells (FIG. 9). Specifically, FACS sorted CD14⁺ monocytes were cultured for 7 days, alone or with CLECL1⁺ or with CLECL1⁻ CLL PF B cells or with 1 μg/ml rCLECL1. At the end of culture, using flow cytometry, the numbers of M1 and M2 macrophages were determined based on presence of CD206, CD80, TNF-alpha, and IL-4. N=8 CLL patients, with each dot in FIG. 9 representing one patient.

Thus, CLECL1⁺ PF cells and rCLECL1 protein can create another immunosuppressive action on the immune system, in addition to the actions of IL-4. Thus, eliminating the cells that bear CLECL1 would benefit patients by correcting two causes for impaired anti-tumor immunity.

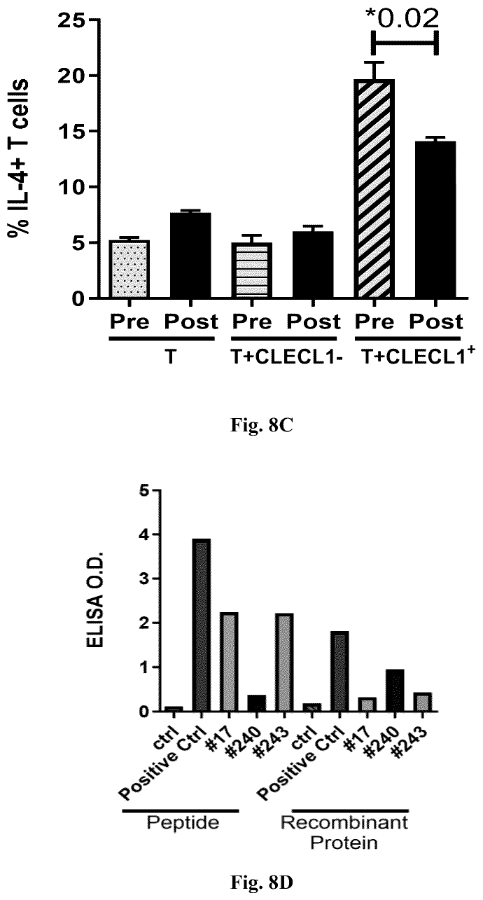

Notably, IL-4 preferentially and significantly improved survival of CLECL1⁺ PF cells but not CLECL1⁻ PF cells (FIG. 7A). This is striking, since the density of IL-4R on CLECL1⁻ cells is much greater than on CLECL1⁺ PF cells (P<0.0001) (FIG. 7B). The discovery that CLECL1⁺ PF cells respond to IL-4 is a key node for therapeutic targeting since it represents a feed-forward process: CLECL1⁺ PF cells generate Th2/IL-4, which preferentially keeps alive CLECL1⁺ PF cells that can repeat the cycle. This process also allows these post-mitotic cells, which could contain new genomic mutations developed at the time of DNA replication in the presence of AID and high levels of ROS, to be selected for survival, not only by new genetic changes but also by superior response to an anti-apoptotic signal, IL-4.

Overall, our ex vivo, in vitro, and in vivo results indicate that CLL cells that express CLECL1, specifically those in the recently-divided fraction, are dangerous to patients for several reasons: [1] Since they just divided, they lead to an increase in clonal number; [2] since they are the only cells in a CLL clone that express AID and are the cells that express the highest levels of ROS, DNA replication took place in an intracellular environment that can promote DNA mutations, which could lead to additional, deleterious genomic aberrations that could lead to disease progression; [3] CLECL1⁺ PF cells engage both Tn and Tmem cells, activate and expand them, and promote their differentiation to Th2 cells that produce IL-4. [4] Consequently, the CLECL1-shaped Th2 TME facilitates the survival and proliferation of tumor cells, in particular those that are CLECL1⁺ which could have developed additional genomic aberrations; [5] CLECL1⁺ PF cells lead to an immunosuppressive TME that blocks the ability of cytolytic T cells to react to and kill the leukemia cells in two ways: by inducing IL-4 and by skewing the monocyte/macrophage populations to M2.

Therefore, CLECL1⁺ PF cells in CLL clones are key targets for therapy. This indicates that eliminating CLECL1⁺ CLL cells or blocking their promotion of an immunosuppressive TME by administering anti-CLECL1 antibodies will improve CLL treatment, especially for those patients with CLL who are resistant to therapy by targeted agents, in particular BTKi. This is especially relevant because of our finding that CLL clones that are resistant to ibrutinib and other BTKi have more CLECL1⁺ PF cells and higher levels of IL-4R and its downstream target phosphoSTAT6 (FIG. 7).

Gene expression analyses indicate that CLECL1 is expressed in activated B cells, DCs, memory B cells, plasmablasts, long-lived plasma cells, and Langerhans cells. Also, CLECL1 can be expressed in cells of the myelo-monocytic lineage. Hence, eliminating or blocking the function of CLECL1⁺ PF cells would have benefit in:
[1] B-cell lymphoproliferative disorder, such as but not limited to CLL, follicular lymphoma, Burkitt's lymphoma, mantle cell lymphoma, marginal zone lymphoma, Waldenstrom's macroglobulinemia, diffuse large B-cell lymphoma, and multiple myeloma;
[2] acute leukemia such as but not limited to acute lymphocytic leukemia, acute myeloid leukemia, or acute myelomonocytic leukemia;
[3] an autoimmune condition where the production of antibodies by B cells or their derivatives, e.g., plasmablasts and plasma cells, are pathogenic, such as but not limited to systemic lupus erythematosus (SLE), rheumatoid arthritis, graft versus host disease (GvHD) or multiple sclerosis;
[4] a disease where the generation of a Th2 microenvironment is detrimental, such as but not limited to atopic dermatitis, asthma, hyper-IgE syndrome, Langerhans cell histiocytosis;
[5] a disease where cells of the granulocyte lineage are pathogenic, such as but not limited to myelodysplastic syndrome (MDS), and chronic myelogenous leukemia (CML).

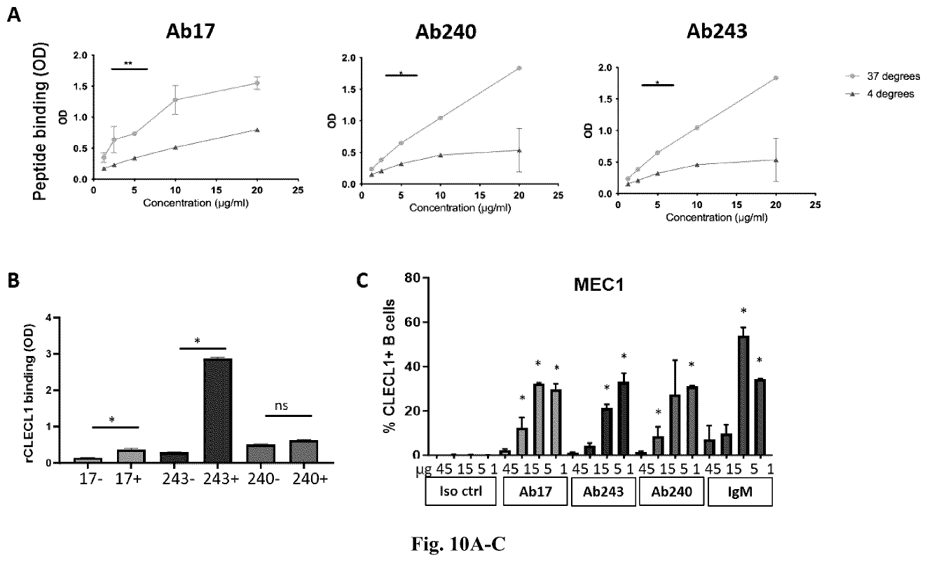

Hybridomas were created from the spleen cells of a mouse immunized with the C-terminal portion of CLECL1-IQDITAMVRENI (SEQ ID NO:3). A cysteine “(C)” amino acid was inserted at the 5′ end of the peptide, adjacent to isoleucine “(I)”, to allow conjugation of the peptide to Keyhole limpet hemocyanin (KLH). Attaching KLH to a small molecule enhances the immunogenicity of the small molecule by eliciting T-cell help, thereby increasing the likelihood of priming murine B cells to the peptide. After several injections of the antigen, initially in complete Freund's adjuvant and subsequently in incomplete Freund's adjuvant, and finally in saline, hybridomas were created by standard technologies. After screening for reactivity with the immunizing antigen (peptide-KLH), the isolated peptide, and the whole length of the external domain of CLECL1, reactivity with CLECL1 displayed on the surface of a B-cell line (MEC1) from a patient with CLL, the animal was sacrificed and spleen cells used to create hybridomas. Based on various cross reactivities (FIG. 10), 3 hybridomas that they secrete mAbs reactive with the 12-mer peptide and rCLECL1 were characterized. These include mAb 17.2 (murine IgG1κ), 243.6 (murine IgG1κ), and 240.29 (murine IgG1λ).

The amino acid sequences for variable (V) regions of the heavy (H) and light (L) chains of the three monoclonal antibodies are shown below.

17.2.3VH
(SEQ ID NO: 4)
EVQLQQSGAELVRSGASVKLSCTTSGFNIKDFYIHWVRQRPEQGL
EWIGWIDPKNDDTEYAPRFQGKATVTADTSSNTAYLHLSGLTSED
TAVYYCNGRYPTMDFWGQGTSVT
17.2.3VL
(SEQ ID NO: 5)
EIVLTQSPITMTTSPGEKITITCSASSSISSHYLHWYQQKPGFSP
KLLIYRASNLASGVPTRFSGSGSGTSYSLTIDTMEAEDVATYFCQ
QGSSLPLTFGAGTKLELK
243.6.1VH
(SEQ ID NO: 6)
EVQLQQSGTVLVRSGASVKLSCTASGFNIKEYYIHWVKQRPEQGL
EWIAWIDLENGESEYAPKFQGKATLTADTSSNTAYLQLSGLTSED
TAVYYCNAFHGNYAHNYAIDYWGQGTSVTVSQ
243.6.1VL
(SEQ ID NO: 7)
DIVMSQSPSSLAVSAGEKVTMTCKSSQSLLNSRTRKNYLAWYQQK
PGQSPKLLIYWASTRKSGVPDRFTGSGSGTDFTLTISSVQAEDLA
VYYCKQSYNLRTFGGGTKLEIK
240.29.3VH
(SEQ ID NO: 8)
EVKLLESGGGLVQPGGSLKLSCAASGFDFSRYWMSWVRQAPGKGL
EWIGEINPDSSTINYTPSLKDKFIISRDNAKNTLYLQMSKVRSED
TALYYCARRGGYFAYWGQGTLVTVSA
240.29.3VL
(SEQ ID NO: 9)
QAVVTQESALTTSPGGTVILTCRSSTGAVTTSNYANWVQEKPDHL
FTGLIGGTSNRAPGVPVRFSGSLIGDKAALTITGAQTEDDAMYFC
ALWYSTHYVFGGGTKVTVL

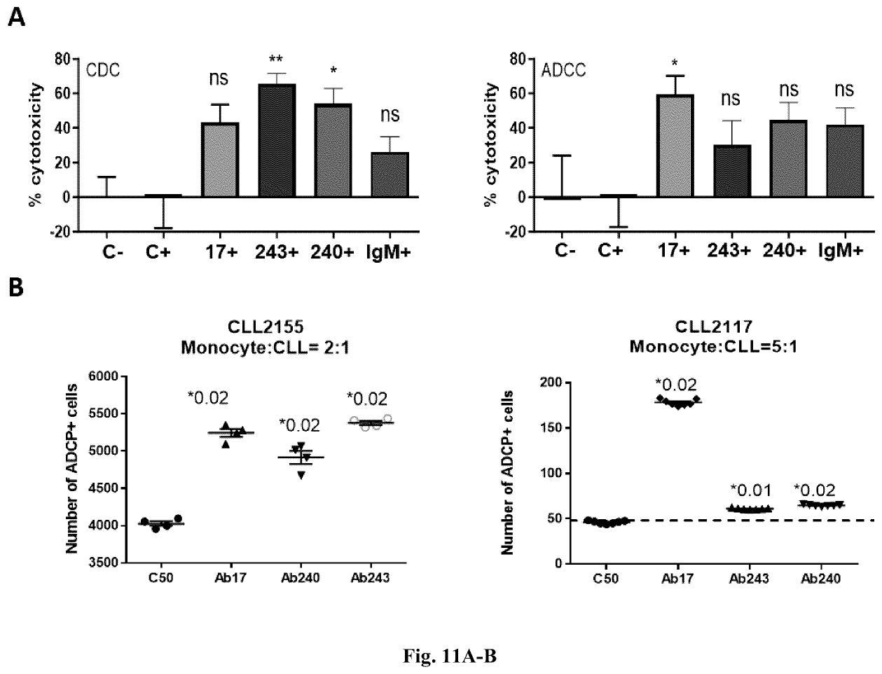

The mAbs produced by these hybridomas kill CLL cells in vitro by at least three effector mechanisms: [1] antibody-dependent cellular cytotoxicity (ADCC); [2] complement-dependent cytotoxicity (CDC); and [3] antibody-dependent cellular phagocytosis (ADCP). The efficiency at which these cytotoxic mechanisms differed based on the specific mAb and its IG heavy chain type (IgG1 vs. IgG3) (FIG. 11).

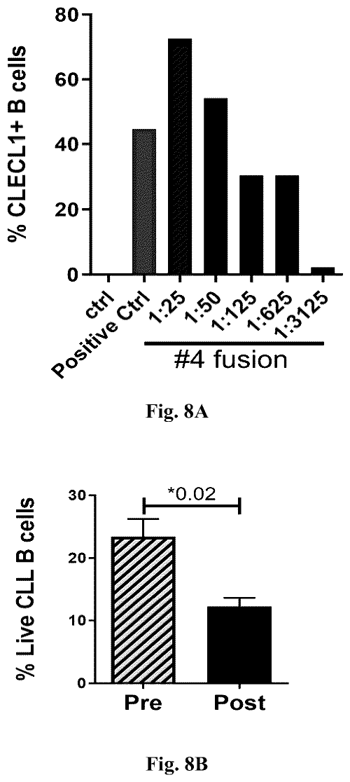

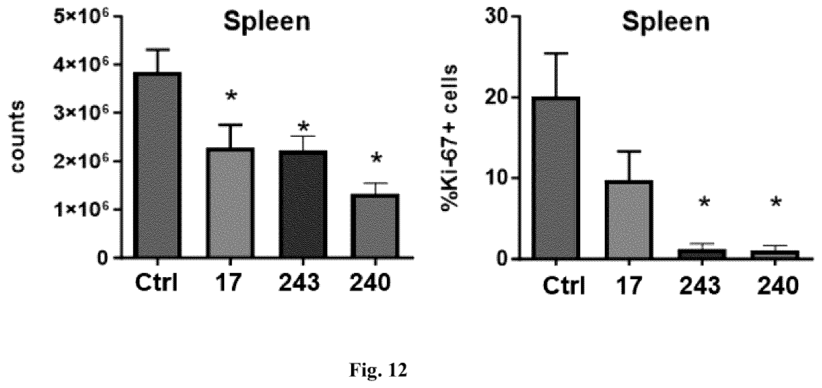

In addition, these mAbs block the ability of CLECL1⁺ PF cells to induce/expand T cells to produce IL-4, illustrating their potential benefit to patients with CLL and possibly patients with other B-lymphoid malignancies, since that could correct the immune deficiency (anti-tumor immunity) that CLECL1⁺ PF cells and rCLECL1 protein can induce (FIGS. 6 and 12).

While illustrative embodiments of the disclosure have been described and illustrated above, it should be understood that these are exemplary of the disclosure and are not to be considered as limiting. Additions, deletions, substitutions, and other modifications can be made without departing from the spirit or scope of the disclosure. Accordingly, the disclosure is not to be considered as limited by the foregoing description.

### CITED PUBLICATIONS

ADDIN EN.REFLIST 1. Furman, R. R. et al. Ibrutinib resistance in chronic lymphocytic leukemia. N Engl J Med 370, 2352-2354 (2014).
2. Ahn, I. E. et al. Clonal evolution leading to ibrutinib resistance in chronic lymphocytic leukemia. Blood 129, 1469-1479 (2017).
3. Woyach, J. A. et al. Resistance mechanisms for the Bruton's tyrosine kinase inhibitor ibrutinib. N Engl J Med 370, 2286-2294 (2014).
4. Ryan, E. J. et al. Dendritic cell-associated lectin-1: a novel dendritic cell-associated, C-type lectin-like molecule enhances T cell secretion of IL-4. J Immunol 169, 5638-5648 (2002).
5. Ryan, E. J. et al. Dendritic Cell-Associated Lectin-1: A Novel Dendritic Cell-Associated, C-Type Lectin-Like Molecule Enhances T Cell Secretion of IL-4. The Journal of Immunology 169, 5638-5648 (2002).
6. Ryan, E. J., Magaletti, D., Draves, K. E. & Clark, E. A. Ligation of dendritic cell-associated lectin-1 induces partial maturation of human monocyte derived dendritic cells. Hum Immunol 70, 1-5 (2009).
7. Calissano, C. et al. Intraclonal complexity in chronic lymphocytic leukemia: fractions enriched in recently born/divided and older/quiescent cells. Mol Med 17, 1374-1382 (2011).
8. Patten, P. E. et al. IGHV-unmutated and IGHV-mutated chronic lymphocytic leukemia cells produce activation-induced deaminase protein with a full range of biologic functions. Blood 120, 4802-4811 (2012).
9. Kluckova, K. et al. B-cell Receptor Signaling Induced Metabolic Alterations in Chronic Lymphocytic Leukemia Can Be Partially Bypassed by TP53 Abnormalities. Hemasphere 6, e722 (2022).
10. Shih-Shih Chen, X.-J. Y., Martina Cardillo, Anita Ng, Andrew Shih, Gonzalo Blanco, Morgan King-Richards, Yu-Ju Chen, Yasmine Kieso, Joanna M. Rhodes, Kanti R Rai, Nicholas Chiorazzi; CLECL1⁺ CLL Cells: A Key Subset That Drives CLL Survival and Proliferation and Shapes a Th2-Biased Tumor Microenvironment. Blood 140, 1528-1529 (2022).
11. Howard, M. et al. Identification of a T cell-derived b cell growth factor distinct from interleukin 2. J Exp Med 155, 914-923 (1982).
12. Herndon, T. M. et al. Direct in vivo evidence for increased proliferation of CLL cells in lymph nodes compared to bone marrow and peripheral blood. Leukemia 31, 1340-1347 (2017).
13. Gine, E. et al. Expanded and highly active proliferation centers identify a histological subtype of chronic lymphocytic leukemia (“accelerated” chronic lymphocytic leukemia) with aggressive clinical behavior. Haematologica 95, 1526-1533 (2010).
14. Lew, T. E. et al. Outcomes of patients with CLL sequentially resistant to both BCL2 and BTK inhibition. Blood Adv (2021).
15. Huntington, S. F. et al. Real-world analysis of adverse event rates after initiation of ibrutinib among Medicare beneficiaries with chronic lymphocytic leukemia. Cancer Med 13, e6953 (2024).
16. Winter, G. & Milstein, C. Man-made antibodies. Nature 349, 293-299 (1991).
17. Morrison, S. L., Johnson, M. J., Herzenberg, L. A. & Oi, V. T. Chimeric human antibody molecules: mouse antigen-binding domains with human constant region domains. Proc Natl Acad Sci USA 81, 6851-6855 (1984).
18. Gross, G., Waks, T. & Eshhar, Z. Expression of immunoglobulin-T-cell receptor chimeric molecules as functional receptors with antibody-type specificity. Proc Natl Acad Sci USA 86, 10024-10028 (1989).
19. Eshhar, Z., Waks, T., Gross, G. & Schindler, D. G. Specific activation and targeting of cytotoxic lymphocytes through chimeric single chains consisting of antibody-binding domains and the gamma or zeta subunits of the immunoglobulin and T-cell receptors. Proc Natl Acad Sci USA 90, 720-724 (1993).
20. Mezosi-Csaplar, M., Szoor, A. & Vereb, G. CD28 and 41BB Costimulatory Domains Alone or in Combination Differentially Influence Cell Surface Dynamics and Organization of Chimeric Antigen Receptors and Early Activation of CAR T Cells. Cancers (Basel) 15 (2023).
21. Cappell, K. M. & Kochenderfer, J. N. A comparison of chimeric antigen receptors containing CD28 versus 4-1BB costimulatory domains. Nat Rev Clin Oncol 18, 715-727 (2021).
22. Ying, Z. et al. Parallel Comparison of 4-1BB or CD28 Co-stimulated CD19-Targeted CAR-T Cells for B Cell Non-Hodgkin's Lymphoma. Mol Ther Oncolytics 15, 60-68 (2019).
23. Porter, D. L., Levine, B. L., Kalos, M., Bagg, A. & June, C. H. Chimeric antigen receptor-modified T cells in chronic lymphoid leukemia. N. Engl. J. Med. 365, 725-733 (2011).
24. Brentjens, R. J. et al. Safety and persistence of adoptively transferred autologous CD19-targeted T cells in patients with relapsed or chemotherapy refractory B-cell leukemias. Blood 118, 4817-4828 (2011).
25. Frey, N. V. & Porter, D. L. CAR T-cells merge into the fast lane of cancer care. Am J Hematol 91, 146-150 (2016).
26. Turtle, C. J. et al. Durable Molecular Remissions in Chronic Lymphocytic Leukemia Treated With CD19-Specific Chimeric Antigen Receptor-Modified T Cells After Failure of Ibrutinib. Journal of clinical oncology: official journal of the American Society of Clinical Oncology 35, 3010-3020 (2017).
27. Romero, D. After ibrutinib, CAR T cells induce responses. Nature Reviews Clinical Oncology 14, 588 (2017).
28. Qi, J., Chen, S. S., Chiorazzi, N. & Rader, C. An IgG1-like bispecific antibody targeting CD52 and CD20 for the treatment of B-cell malignancies. Methods 154, 70-76 (2019).
29. Trabolsi, A., Arumov, A. & Schatz, J. H. Bispecific antibodies and CAR-T cells: dueling immunotherapies for large B-cell lymphomas. Blood Cancer J 14, 27 (2024).
30. Rawstron, A. C. et al. A complementary role of multiparameter flow cytometry and high-throughput sequencing for minimal residual disease detection in chronic lymphocytic leukemia: an European Research Initiative on CLL study. Leukemia 30, 929-936 (2016).
31. Dowling, A. K., Liptrot, S. D., O'Brien, D. & Vandenberghe, E. Optimization and 31. Validation of an 8-Color Single-Tube Assay for the Sensitive Detection of Minimal Residual Disease in B-Cell Chronic Lymphocytic Leukemia Detected via Flow Cytometry. Lab Med 47, 103-111 (2016).

What is claimed is:

1. A method of treating a disease in a patient in need thereof comprising administrating to the patient a therapeutically effective amount of an anti-CLECL1 antibody or antigen-binding fragment thereof,
wherein the disease is one or more of chronic lymphocytic leukemia (CLL), a B-cell lymphoproliferative disorder, follicular lymphoma, Burkitt's lymphoma, mantle cell lymphoma, marginal zone lymphoma, Waldenstrom's macroglobulinemia, diffuse large B-cell lymphoma, multiple myeloma; acute leukemia, acute lymphocytic leukemia, acute myeloid leukemia, acute myelomonocytic leukemia, an autoimmune condition where the production of antibodies by B cells or their derivatives are pathogenic, systemic lupus erythematosus (SLE), rheumatoid arthritis, graft versus host disease (GvHD), multiple sclerosis, a disease where the generation of a Th2 microenvironment is detrimental, atopic dermatitis, asthma, hyper-IgE syndrome, Langerhans cell histiocytosis, a disease where cells of the granulocyte lineage are pathogenic, myelodysplastic syndrome (MDS), and chronic myelogenous leukemia (CML).

2. The method of claim 1, wherein the antibody or fragment thereof is administered as a component of a Chimeric Antigen Receptor (CAR) T cell or as a bi- or tetra-specific antibody that co-ligates T cells or NK cells and abnormal cells in the disease or as a bi- or tetra-specific antibody that binds only the B cells in CLL or only abnormal cells in the disease.

3. The method of claim 1, wherein the disease is chronic lymphocytic leukemia (CLL).

4. The method of claim 3, wherein the patient is resistant to treatment with an inhibitor of Bruton's tyrosine kinase (BTKi).

5. The method of claim 1, wherein the antibody is a monoclonal antibody.

6. The method of claim 1, wherein the antibody or fragment thereof is chimeric or humanized.

7. The method of claim 1, wherein the antibody is an IgG monoclonal antibody.

8. The method of claim 1, wherein the antibody is an antagonistic monoclonal antibody.

9. The method of claim 1, wherein the antibody is a monoclonal antibody that binds to amino acid sequence DWKVHKGKCY WIAETKKSWN KSQNDCAINN SYLMVIQDIT AMVRENI (SEQ ID NO:2).

10. The method of claim 1, wherein the antibody is a monoclonal antibody that binds to amino acid sequence IQDITAMVRENI (SEQ ID NO:3).

11. The method of claim 1, wherein the antibody is a monoclonal antibody comprising:
a) a heavy chain complementarity determining region (CDR) comprising SEQ ID NO:4, EVQLQQSGAELVRSGASVKLSCTTSGFNIKDFYIHWVRQRPEQGLEWIGWIDPKNDDTE YAPRFQGKATVTADTSSNTAYLHLSGLTSEDTAVYYCNGRYPTMDFWGQGTSVT, and a light chain CDR comprising SEQ ID NO:5, EIVLTQSPITMTTSPGEKITITCSASSSISSHYLHWYQQKPGFSPKLLIYRASNLASGVPTRF SGSGSGTSYSLTIDTMEAEDVATYFCQQGSSLPLTFGAGTKLELK; or
b) a heavy chain CDR comprising SEQ ID NO:6, EVQLQQSGTVLVRSGASVKLSCTASGFNIKEYYIHWVKQRPEQGLEWIAWIDLENGESE YAPKFQGKATLTADTSSNTAYLQLSGLTSEDTAVYYCNAFHGNYAHNYAIDYWGQGTS VTVSQ, and a light chain CDR comprising SEQ ID NO:7, DIVMSQSPSSLAVSAGEKVTMTCKSSQSLLNSRTRKNYLAWYQQKPGQSPKLLIYWAST RKSGVPDRFTGSGSGTDFTLTISSVQAEDLA VYYCKQSYNLRTFGGGTKLEIK; or
c) a heavy chain CDR comprising SEQ ID NO:8, EVKLLESGGGLVQPGGSLKLSCAASGFDFSRYWMSWVRQAPGKGLEWIGEINPDSSTIN YTPSLKDKFIISRDNAKNTLYLQMSKVRSEDTALYYCARRGGYFAYWGQGTLVTVSA, and a light chain CDR comprising SEQ ID NO:9, DIVLTQSPASLAVSLGQRATISYRASKSVSTSGYSYMHWNQQKPGQPPRLLIYLVSNLES GVPARFSGSGSGTDFTLNIHPVEEEDAATYYCQHIRELTRSEGGPSWK.

12. The method of claim 1, wherein the antibody or fragment thereof is administered to the patient by intravenous injection.

13. The method of claim 1, wherein the patient is a human or a veterinary animal.

14. A method for predicting disease relapse in a patient or for measuring residual disease in a patient, the method comprising analyzing a sample from blood, bone marrow or a lymph node of a patient who is or has been treated for the disease for the presence of CLECL1⁺ cells,
wherein the presence of CLECL1+ cells in the patient's sample is detected using a monoclonal antibody to CLECL1 or antigen-binding fragment thereof,
wherein appearance of CLECL1⁺ cells in the sample is indicative of development of resistance to therapy or presence of residual disease, and
wherein the disease is one or more of chronic lymphocytic leukemia (CLL), a B-cell lymphoproliferative disorder, follicular lymphoma, Burkitt's lymphoma, mantle cell lymphoma, marginal zone lymphoma, Waldenstrom's macroglobulinemia, diffuse large B-cell lymphoma, multiple myeloma; acute leukemia, acute lymphocytic leukemia, acute myeloid leukemia, acute myelomonocytic leukemia, an autoimmune condition where the production of antibodies by B cells or their derivatives are pathogenic, systemic lupus erythematosus (SLE), rheumatoid arthritis, graft versus host disease (GvHD), multiple sclerosis, a disease where the generation of a Th2 microenvironment is detrimental, atopic dermatitis, asthma, hyper-IgE syndrome, Langerhans cell histiocytosis, a disease where cells of the granulocyte lineage are pathogenic, myelodysplastic syndrome (MDS), and chronic myelogenous leukemia (CML).

15. A monoclonal antibody to CLECL1 comprising:
a) a heavy chain complementarity determining region (CDR) comprising SEQ ID NO:4, EVQLQQSGAELVRSGASVKLSCTTSGFNIKDFYIHWVRQRPEQGLEWIGWIDPKNDDTE YAPRFQGKATVTADTSSNTAYLHLSGLTSEDTAVYYCNGRYPTMDFWGQGTSVT, and a light chain CDR comprising SEQ ID NO:5, EIVLTQSPITMTTSPGEKITITCSASSSISSHYLHWYQQKPGFSPKLLIYRASNLASGVPTRF SGSGSGTSYSLTIDTMEAEDVATYFCQQGSSLPLTFGAGTKLELK; or
b) a heavy chain CDR comprising SEQ ID NO:6, EVQLQQSGTVLVRSGASVKLSCTASGFNIKEYYIHWVKQRPEQGLEWIAWIDLENGESE YAPKFQGKATLTADTSSNTAYLQLSGLTSEDTAVYYCNAFHGNYAHNYAIDYWGQGTS VTVSQ, and a light chain CDR comprising SEQ ID NO:7, DIVMSQSPSSLAVSAGEKVTMTCKSSQSLLNSRTRKNYLAWYQQKPGQSPKLLIYWAST RKSGVPDRFTGSGSGTDFTLTISSVQAEDLAVYYCKQSYNLRTFGGGTKLEIK; or
c) a heavy chain CDR comprising SEQ ID NO:8, EVKLLESGGGLVQPGGSLKLSCAASGFDFSRYWMSWVRQAPGKGLEWIGEINPDSSTIN YTPSLKDKFIISRDNAKNTLYLQMSKVRSEDTALYYCARRGGYFAYWGQGTLVTVSA, and a light chain CDR comprising SEQ ID NO:9, DIVLTQSPASLAVSLGQRATISYRASKSVSTSGYSYMHWNQQKPGQPPRLLIYLVSNLES GVPARFSGSGSGTDFTLNIHPVEEEDAATYYCQHIRELTRSEGGPSWK.

16. The monoclonal antibody of claim 15, wherein the monoclonal antibody is chimeric or humanized.

17. The monoclonal antibody of claim 15, wherein the monoclonal antibody is labeled with a fluorescent, radioactive, or enzyme label, or with a cytotoxin.

18. The monoclonal antibody of claim 15, wherein the monoclonal antibody is a component of a CAR-T cell, or a bi- or tetra-specific antibody that binds T cells and CLECL1 on target cells or a bi- or tetra-specific antibody that binds an antigen on the surface of a target cell as well as binding CLECL1.
